(19) United States
(12) Patent Application Publication
(10) Pub. No.: US 2010/0234927 A1
(43) Pub. Date: Sep. 16, 2010

(54) LIGHTING APPARATUS OF FACIAL AND LIGHTING METHOD THEREOF

(76) Inventor: Mei-Chun Lin, Sindian City (TW)

Correspondence Address: KILE GOEKJIAN REED & MCMANUS, 1200 NEW HAMPSHIRE AVE, NW, SUITE 570, WASHINGTON, DC, 20036, US

(21) Appl. No.: 12/401,914

(22) Filed: Mar. 11, 2009

(51) Int. Cl. A61N 5/06

(52) U.S. Cl. 607/91

(57) ABSTRACT

A lighting apparatus of facial includes a first silica gel layer, a control unit disposed on the first silica gel layer, a lighting module and a second silica gel layer. The lighting module is disposed on the first silica gel layer and includes a plurality of lighting units corresponding to purlieus between a forehead and a nose, a periphery of two eyes, cheeks and a periphery of a mouth of the first silica gel layer. The lighting units are coupled to the control unit. The second silica gel layer is combined with the first silica gel layer to package the control unit and the lighting module. Accordingly, the control unit controls the lighting units corresponding to the purlieus between the forehead and the nose, the periphery of the two eyes, the cheeks and the periphery of the mouth to generate pulsed light with desired wavelength.

### BACKGROUND OF THE INVENTION

1. Field of the Invention

The present invention relates to a lighting apparatus of facial and lighting method thereof, and more particularly to a lighting apparatus of facial and lighting method thereof which can provide the desired facial light for facial locations according to facial demands of every facial location.

2. Description of Related Art

With the development of people's living standards, people pay more and more attention to their health and facial. So there are more and more health and facial methods on the market, for example, practical health foods, or daubing healthcare products on people's faces so that the faces are in the optimum state, or injecting botulinus or hyaluronic acid into special locations to eliminate facial wrinkles. Furthermore, at present, some people improve their skin conditions and qualities via light waves to achieve the effect of people's skin care.

Light with special wavelength irradiating skin to treat and care skin or improve skin qualities is an important achievement of current medical treatment technologies, which is a low energy photon therapy (LEPT). Many advanced medical researches prove that light in different colors irradiating human cells will bring different efficacies. For example, red light (the wavelength is 630-740 nm) can improve cell metabolism so as to accelerate wound healing, and can relief throes from inflammation during the earlier and the middle inflammation periods and treat pimples. Blue light (the wavelength is 400-445 nm) can be absorbed by porphyria of acne bacteria to destroy bacteria, thereby achieving the sterilization effect; and the blue light can stimulate parasympathetic nervous system to relief anxiety and hostility. Further, a far infrared ray (the wavelength is more than 3000 nm) can activate histiocytes and accelerate blood circulation, and accelerate nutrition and enzymes supply to promote metabolism, and provide the effects of deodorization, dryness, dehumidification and sterilization besides immunity enhancement.

Human facial mainly focuses on faces. General face facial mainly focuses on eliminating pimples and wrinkle and whiting skin. At present, multi-function facial and skin treating instruments are always used to beautify human faces on the market. During operation, a light emitting cover covers a face firstly, and then the facial is executed on the face according to the set light, intensity and time.

However, during use of the beforementioned multi-function facial and skin treating instruments, users must stay at a special location to execute the face facial, so it is very inconvenient. Additionally, the multi-function facial and skin treating instruments can only provide a kind of preset light at the same time to achieve a kind of corresponding facial effect, so they cannot meet the different facial demands for different face locations.

### SUMMARY OF THE INVENTION

An object of the present invention is to provide a lighting apparatus of facial and lighting method thereof wherein the lighting apparatus of facial can cover a user's face and provide corresponding facial light for the user's different face locations thereby achieving the desired facial effects.

To achieving the above-mentioned object, a lighting apparatus of facial in accordance with a preferred embodiment of the present invention is provided. The lighting apparatus of facial includes a first silica gel layer; a control unit disposed on the first silica gel layer; a lighting module which is disposed on the first silica gel layer and includes a plurality of lighting units corresponding to purlieus between a forehead and a nose, a periphery of two eyes, cheeks and a periphery of a mouth of the first silica gel layer, wherein the plurality of lighting units are coupled to the control unit; and a second silica gel layer which has a control unit receiving room and a plurality of lighting unit receiving rooms and is combined with the first silica gel layer to package the control unit and the lighting module, wherein the receiving rooms of the second silica gel layer respectively receive the control unit and the plurality of lighting units; whereby the control unit controls the lighting units corresponding to the purlieus between the forehead and the nose, the periphery of the two eyes, the cheeks and the periphery of the mouth to generate pulsed light with desired wavelength.

To achieving the above-mentioned object, a lighting method in accordance with the present invention is further provided. The lighting method includes the steps of: firstly choosing locations of the lighting apparatus of facial desired to be lighted, which include the purlieus between the forehead and the nose, the periphery of the two eyes, the cheeks and the periphery of the mouth; and then controlling the lighting units corresponding to the chosen locations to emit light according the chose locations.

Consequently, the lighting apparatus of facial of the present invention can directly cover users' faces so that the users can accept the facial lighting of the lighting apparatus of facial whenever and wherever, so the lighting apparatus of facial is easy to be carried, which avoids the status that users must stay at a special location to accept the facial lighting, thereby the lighting apparatus of facial is convenient for use.

Also, the lighting method of the present invention can provide corresponding facial light for the user's different face locations to achieve the desired facial effects, thereby breaking through the use limitation of conventional multi-function facial and skin treating instruments.

To further understand other objects and advantages of the present invention, please refer to the following detailed description and drawings related the present invention. However, the drawings are only to be used as references and explanations, not to limit the present invention.

### BRIEF DESCRIPTION OF THE DRAWINGS

FIG. 1 is a schematic view of a first silica gel layer of a preferred embodiment of the present invention;

FIG. 2 is a schematic view of a second silica gel layer of the preferred embodiment of the present invention;

FIG. 3 is a functional block diagram of a control unit of the present invention;

FIG. 4 is a circuit schematic view of the control unit of the present invention; and

FIG. 5 is a circuit schematic view of lighting units of the present invention.

### DETAILED DESCRIPTION OF THE PREFERRED EMBODIMENTS

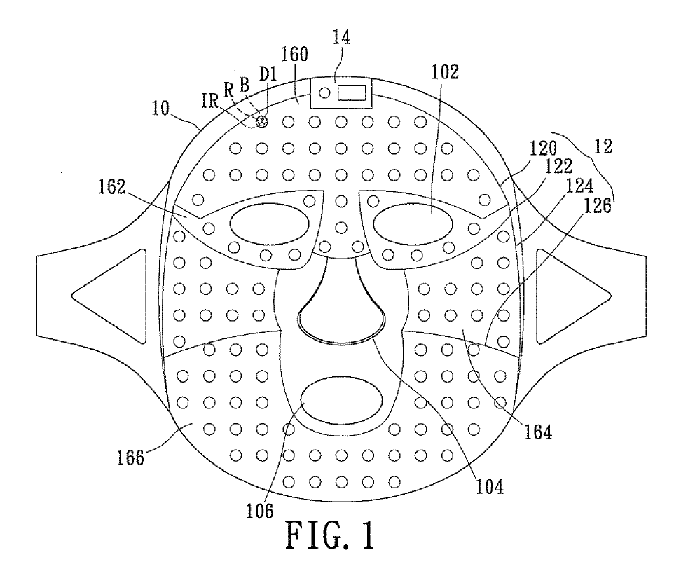

Please refer to FIG. 1 illustrating a first silica gel layer of a preferred embodiment according to the present invention. As shown in FIG. 1, the material of the first silica gel layer 10 may be nontransparent silica gel in any color, or may be any nontransparent soft polyurethane (PU) instead. The first silica gel layer 10 has an inner face and an outer face, wherein the inner face is used for the assembly of a control unit 14 and a lighting module 12 such as a lighting soft circuit board or a lighting bar, and the outer face of the first silica gel layer 10 is used for contacting the external during use.

Refer to FIG. 1 again, the first silica gel layer 10 has double-eye openings 102, a nose opening 104 and a mouth opening 106. Father, the control unit 14 is disposed on the first silica gel layer 10. The lighting module 12 is disposed on the first silica gel layer 10 and includes a first partial lighting module 120, a second partial lighting module 122, a third partial lighting module 124 and a fourth partial lighting module 126, wherein the first partial lighting module 120 is disposed on the purlieus between the forehead and the nose of the first silica gel layer 10, the second partial lighting module 122 is disposed the periphery of the two eyes of the first silica gel layer 10, the third partial lighting module 124 is disposed on the cheeks of the first silica gel layer 10, and the fourth partial lighting module 126 is disposed on the periphery of the mouth of the first silica gel layer 10.

Refer to FIG. 1 again, the first partial lighting module 120 has a first lighting unit 160, the second partial lighting module 122 has a second lighting unit 162, the third partial lighting module 124 has a third lighting unit 164, and the fourth partial lighting module 160 has a fourth lighting unit 166. The before mentioned lighting units 160, 162, 164, 166 are all coupled to the control unit 14.

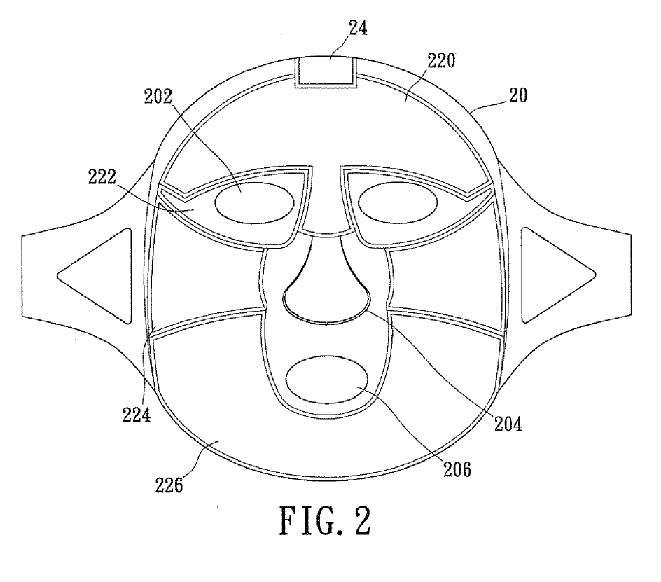

Please refer to FIG. 2, cooperating with FIG. 1. FIG. 2 is a schematic view of a second silica gel layer of the preferred embodiment according to the present invention. As shown in FIG. 2, the second silica gel layer 20 of the present invention has double-eye openings 202, a nose opening 204 and a mouth opening 206, corresponding to the first silica gel layer 10. The second silica gel layer 20 of the present invention further has a control unit receiving room 24 and a plurality of lighting unit receiving rooms 220, 222, 224, 226.

Please refer to FIG. 1 and FIG. 2, the lighting apparatus of facial of the present invention is formed by combining the second silica gel layer 20 with the first silica gel layer 10 via a joining element (not labeled). After combination, the second silica gel layer 20 may package the control unit 14 and the lighting module 12 disposed on the first silica gel layer 10. The control unit receiving room 24 of the second silica gel layer 20 may receive the control unit 14, the first lighting unit receiving room 220 may receive the first lighting unit 160 of the first partial lighting module 120, the second lighting unit receiving room 222 may receive the second lighting unit 162 of the second partial lighting module 122, the third lighting unit receiving room 224 may receive the third lighting unit 164 of the third partial lighting module 124, and the fourth lighting unit receiving room 226 may receive the fourth lighting unit 166 of the fourth partial lighting module 126.

Refer to FIG. 1 and FIG. 2 again. Users can operate the control unit 14 of the lighting apparatus of facial of the present invention to control the plurality of lighting units 160, 162, 164, 166 to generate the pulsed light with desired wavelength for the purlieus between the forehead and the nose, the periphery of the two eyes, the cheeks and the periphery of the mouth. The lighting units 160, 162, 164, 166 all include a plurality of lighting elements D1 each of which is an encapsulated piece of blue light B, red light R and far infrared ray IR.

The control unit 14 may control each lighting unit 160, 162, 164, 166 to provide three-wavelength light which includes red light R, blue light B and far infrared ray IR. Alternatively, the control unit 14 may control each lighting unit 160, 162, 164, 166 to provide double-wavelength light which includes any two kinds of light of red light R, blue light B and far infrared ray IR. Alternatively, the control unit 14 may control each lighting unit 160, 162, 164, 166 to provide single-wavelength light which may be red light R, blue light B or far infrared ray IR.

Additionally, users may operate the control unit 14 to set the desired control modes, which include a full-lighting mode, a lighting mode for the purlieus between the forehead and the nose, a double-eye lighting mode, a cheek lighting mode, a mouth lighting mode and so on. At the same time, users may also set the lighting time and the lighting intensity via operating the control unit 14.

Refer to FIG. 1 and FIG. 2 again, the following describes the corresponding light and facial effects generated by the control unit 14 in the various control modes. When working under the lighting mode for the purlieus between the forehead and the nose, the control unit 14 controls the first lighting unit 160 corresponding to the purlieus between the forehead and the nose of the first silica gel layer 10 to generate the red light (the wavelength is 630-740 nm) and the far infrared ray (the wavelength is more than 3000 nm) for drying, dehumidifying and sterilizing the forehead, thereby treating pimples.

Also, when working under the double-eye lighting mode, the control unit 14 controls the second lighting unit 162 corresponding to the periphery of the two eyes of the first silica gel layer 10 to generate the red light (the wavelength is 630-740 nm) and the far infrared ray (the wavelength is more than 3000 nm) for cell activation of the periphery of the two eyes, thereby achieving the effect of wrinkle removal.

When working in the cheek lighting mode, the control unit 14 controls the third lighting unit 164 corresponding to the cheeks of the first silica gel layer 10 to generate the red light (the wavelength is 630-740 nm) and the far infrared ray (the wavelength is more than 3000 nm) for drying, dehumidifying and sterilizing the nose, thereby treating pimples.

When working in the mouth lighting mode, the control unit 14 controls the fourth lighting unit 166 corresponding to the periphery of the mouth of the first silica gel layer 10 to generate the red light (the wavelength is 630-740 nm), the far infrared ray (the wavelength is more than 3000 nm) and the blue light (the wavelength is 400-445 nm) for the dephlogistication, dryness, dehumidification and sterilization of the periphery of the mouth, thereby achieving the effect of pimple treatment.

When working in the full-lighting mode, the control unit 14 controls all the lighting units 160, 162, 164, 166 of the first silica gel layer 10 to generate the red light (the wavelength is 630-740 nm), the far infrared ray (the wavelength is more than 3000 nm) and the blue light (the wavelength is 400-445 nm) for the wrinkle removal, dephlogistication, dryness, dehumidification and sterilization of the face, enhancement of immunity, deodorization, anxiety and hostility reduction.

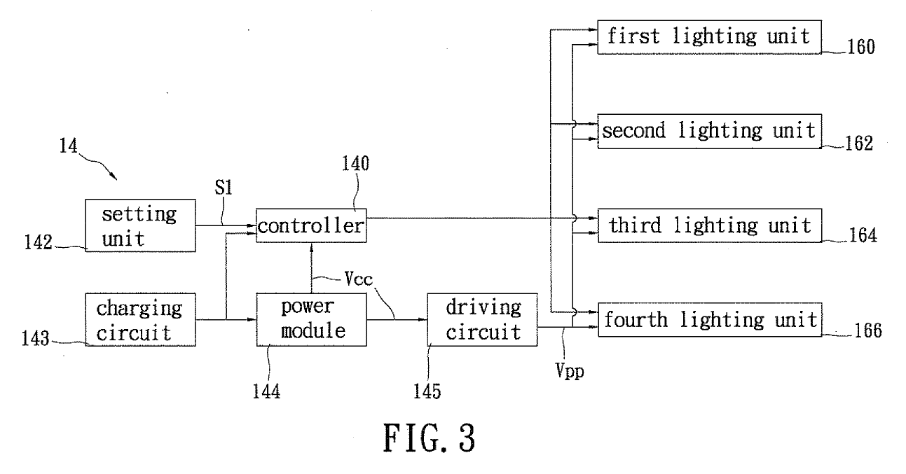

Please refer to FIG. 3 which is a functional block diagram of a control unit of the present invention. The control unit 14 of the present invention is coupled to the first lighting unit 160, the second lighting unit 162, the third lighting unit 164 and the fourth lighting unit 166 and controls the lighting units 160, 162, 164, 166 to generate light, thereby achieving the facial effect of the face locations. The control unit 14 includes a controller 140, a setting unit 142, a charging circuit 143, a driving circuit 145 and a power module 144 such as a secondary battery. The controller 140 is coupled to the setting unit 142, the power module 144, the charging circuit 143, and the above described lighting units 160-166. Further, the driving circuit 145 is coupled to the power module 144 and the above described lighting units 160-166.

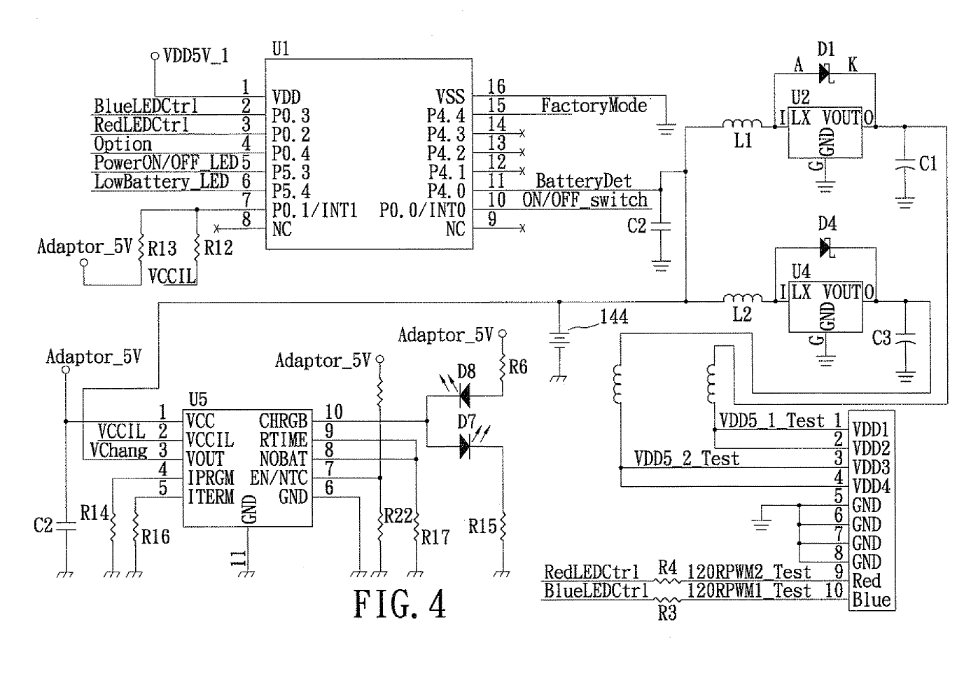

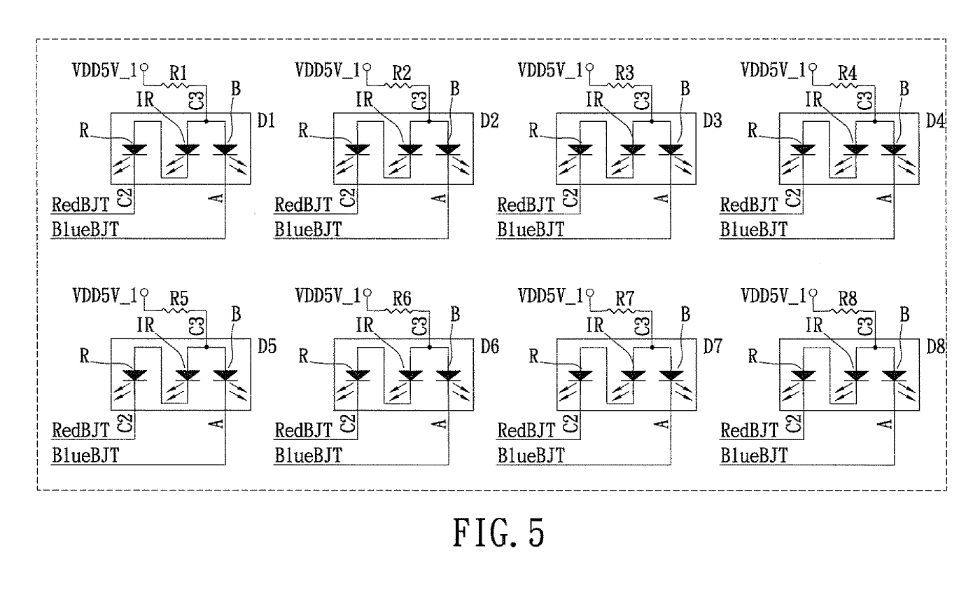

Cooperating with FIG. 4 and FIG. 5, the controller 140 described above takes the integrated circuit U1 of which the model number is SONIX as an example. The charging circuit 143 described above takes the integrated circuit U5 of which the model number is SC805A as an example. The driving circuit 145 described above takes the integrated circuits U1, U4 of which the model number is LD7280 as an example. Also, the lighting units 160-166 all include a plurality of lighting elements D1, D2 . . . DN, each of which is an encapsulated piece of blue light B, red light R and far infrared ray IR.

Refer to FIG. 3 again, based on the set operation for the setting unit 142, the setting unit 142 transmits a set signal S1 to the controller 140. The controller 140 works in the various lighting modes such as the full-lighting mode, the lighting mode for the purlieus between the forehead and the nose, the double-eye lighting mode, the cheek lighting mode and the mouth lighting mode to control the corresponding lighting units 160-166 to emit light according to the set signal S1. The controller 140 may also control the lighting time and the lighting intensity of the full-lighting mode, the lighting mode for the purlieus between the forehead and the nose, the double-eye lighting mode, the cheek lighting mode and the mouth lighting mode according to the set signal S1.

Refer to FIG. 3 again. In the control unit 14, the power module 144 provides a work voltage Vcc for the controller 140 so that the controller can work normally. At the same times the driving circuit 145 boosts the work voltage Vcc from the power module 144, so as to provide a supply voltage Vpp for each lighting unit 160-166 as the power source of each the lighting unit 160-166.

Refer to FIG. 3 again. The charging circuit 143 may be connected with an external power adapter to get a power source from the external. The charging unit 143 charges the power module 144 with the power source. When the charging circuit 143 charges the power module 144, the controller 140 detects that the power module 144 is in the charging state. At this time, the controller 140 will stop performing the operation in the various lighting modes such as the full-lighting mode, the lighting mode for the purlieus between the forehead and the nose, the double-eye lighting mode, the cheek lighting mode and the mouth lighting mode, and stop controlling the lighting time and the lighting intensity of the full-lighting mode, the lighting mode for the purlieus between the forehead and the nose, the double-eye lighting mode, the cheek lighting mode and the mouth lighting mode.

Refer to FIG. 3 again. Users can choose the face locations of the lighting apparatus of facial desired to be lighted, which include the purlieus between the forehead and the nose, the periphery of the two eyes, the cheeks and the periphery of the mouth, via the setting unit 142. Then, based on the operation of the setting unit 142, users can control the lighting units 160-166 corresponding to the chosen locations to emit light and emit light for a preset time, thereby achieving the facial effects of the face locations. Also, users can choose a single face location or a plurality of face locations desired to be lighted according to the demands, thereby saving energy. Accordingly, comparing with conventional lighting apparatus, the present invention can save electric energy under the full-lighting greatly.

Consequently, the lighting apparatus of facial of the present invention can directly cover users' faces so that the users can accept the facial lighting of the lighting apparatus of facial whenever and wherever, so the lighting apparatus of facial is easy to be carried, which avoids the status that users must stay at a special location to accept the facial lighting, thereby the lighting apparatus of facial is convenient for use.

Also, the lighting method of the present invention can provide corresponding facial light for the user's different face locations to achieve the desired facial effects, thereby breaking through the use limitation of conventional multi-function facial and skin treating instruments and avoiding energy waste.

What are disclosed above are only the specification and the drawings of the preferred embodiment of the present invention and it is therefore not intended that the present invention be limited to the particular embodiment disclosed. It will be understood by those skilled in the art that various equivalent changes may be made depending on the specification and the drawings of the present invention without departing from the scope of the present invention.

What is claimed is:

1. A lighting apparatus of facial, comprising:
a first silica gel layer;
a control unit, disposed on the first silica gel layer;
a lighting module, disposed on the first silica gel layer and including a plurality of lighting units corresponding to purlieus between a forehead and a nose, a periphery of two eyes, cheeks and a periphery of a mouth of the first silica gel layer, the plurality of lighting units coupled to the control unit; and
a second silica gel layer, having a control unit receiving room and a plurality of lighting unit receiving rooms, the second silica gel layer combined with the first silica gel layer to package the control unit and the lighting module;
whereby the control unit controls the lighting units corresponding to the purlieus between the forehead and the nose, the periphery of the two eyes, the cheeks and the periphery of the mouth to generate pulsed light with desired wavelength.

2. The lighting apparatus of facial as claimed in claim 1, wherein the control unit receiving room is used for receiving the control unit, and the plurality of lighting unit receiving rooms respectively correspondingly receiving the plurality of lighting units.

3. The lighting apparatus of facial as claimed in claim 2, wherein the lighting module includes:
a first partial lighting module, having a first lighting unit, corresponding to the purlieus between the forehead and the nose of the first silica gel layer;
a second partial lighting module, having a second lighting unit, corresponding to the periphery of the two eyes of the first silica gel layer;
a third partial lighting module, having a third lighting unit, corresponding to the cheeks of the first silica gel layer; and
a fourth partial lighting module, having a fourth lighting unit, corresponding to the periphery of the mouth of the first silica gel layer.

4. The lighting apparatus of facial as claimed in claim 2, wherein the control unit respectively controls the plurality of lighting units to provide three-wavelength light which includes red light, blue light and far infrared ray.

5. The lighting apparatus of facial as claimed in claim 2, wherein the control unit respectively controls the plurality of lighting units to provide double-wavelength light which includes any two kinds of light of red light, blue light and far infrared ray.

6. The lighting apparatus of facial as claimed in claim 2, wherein the control unit respectively controls the plurality of lighting units to provide single-wavelength light which includes red light, blue light or far infrared ray.

7. The lighting apparatus of facial as claimed in claim 1, wherein the lighting unit includes a plurality of lighting elements each of which is an encapsulated piece of blue light, red light and far infrared ray.

8. The lighting apparatus of facial as claimed in claim 1, wherein the control unit includes:
a setting unit, providing a set signal;
a power module, providing a work voltage;
a driving circuit, coupled to the power module and the plurality of lighting units, boosting the work voltage and providing a supply voltage for the plurality of lighting units;
a charging circuit, coupled to the power module and charging the power module with a power source; and
a controller, coupled to the setting unit, the power module, the charging circuit and the plurality of lighting units and performing operations in a full-lighting mode, a lighting mode for the purlieus between the forehead and the nose, a double-eye lighting mode, a cheek lighting mode and a mouth lighting mode to control the corresponding lighting units to emit light according to the set signal.

9. The lighting apparatus of facial as claimed in claim 8, wherein the controller controls lighting time and lighting intensity of the full-lighting mode, the lighting mode for the purlieus between the forehead and the nose, the double-eye lighting mode, the cheek lighting mode and the mouth lighting mode according to the set signal.

10. The lighting apparatus of facial as claimed in claim 9, wherein the controller stops performing the operations in the all lighting modes when the power module is charged.

11. A lighting method of the lighting apparatus of facial as claimed in claim 1, comprising the steps of:
a. choosing locations of the lighting apparatus of facial desired to be lighted, which include the purlieus between the forehead and the nose, the periphery of the two eyes, the cheeks and the periphery of the mouth; and
b. controlling the lighting units corresponding to the chosen locations to emit light according the chose locations.

12. The lighting method as claimed in claim 11, wherein the step b further includes controlling the lighting units corresponding to the chosen locations to emit light according to the chosen locations and a preset time.

13. The lighting method as claimed in claim 12, wherein the step b includes controlling the lighting units to generate three-wavelength light which includes red light, blue light and far infrared ray.

14. The lighting method as claimed in claim 12, wherein the step b includes controlling the lighting units to generate double-wavelength light which includes any two kinds of light of red light, blue light and far infrared ray.

15. The lighting method as claimed in claim 12, wherein the step b includes controlling the lighting units to generate three-wavelength light which includes red light, blue light or far infrared ray.
